(19) United States
(12) Patent Application Publication
(10) Pub. No.: US 2025/0135286 A1
(43) Pub. Date: May 1, 2025

(54) Tibial shock absorption apparatus and methods

(71) Applicant: Evolve Patents PTY PTD, Docklands, Victoria (AU)

(72) Inventor: Luke James Pickett, Byron Bay (AU)

(73) Assignee: Evolve Patents PTY PTD, Docklands, Victoria (AU)

(21) Appl. No.: 18/685,181

(22) PCT Filed: Aug. 22, 2021
(86) PCT No.: PCT/AU2022/050938

§ 371 Date: Feb. 20, 2024

(30) Foreign Application Priority Data
Aug. 20, 2021 (AU) 2021902625
Aug. 10, 2022 (AU) 2022902246

(51) Int. Cl. A63B 24/00; A63B 71/06; G06F 3/01; G16H 40/67

(52) U.S. Cl. CPC A63B 24/0075; A63B 24/0062; A63B 71/0622; G06F 3/011; G16H 40/67; A63B 2071/0625; A63B 2071/0655; A63B 2220/833

(57) ABSTRACT

Apparatus and methods related to tibial shock absorption data collection and processing include a sensory unit for collecting shock absorption data representing shock absorption by hard tissues of the user. data processing and storage devices, housing units and attachment means for attaching sensors to body parts such as the tibia. The collected and processed data is used for coaching the user to optimise muscles usage for shock optimisation for purposes such as injury minimisation, bone density increase and weight loss.

### TECHNICAL FIELD

The present invention relates to an apparatus and method for monitoring and optimising shock absorption during ambulation and other weight bearing activities. Some embodiments of the invention may find application in: the treatment, management and prevention of musculoskeletal injuries and/or pain, such as osteoarthritis; weight loss; and in optimising the lower limb function of walking and running.

### BACKGROUND

Walking and running are daily activities which can have a functional, sporting or leisure purpose. These activities contribute to the wear and tear on the human body over time. Shock absorption during the daily activity of ambulation (be it walking or running) is generally not as efficient as possible. The cause of this inefficiency has not been conclusively determined, but could be contributed to by the advent of running shoes, which provide shock absorption that actually contributes to this inefficiency of gait. It is thought that this external shock absorption potentially provides the body and brain with an opportunity to decrease their own shock absorption capabilities and rely on the external shock absorption of running shoes.

Another theory suggests that evolution has increased the efficiency of the ambulation process in terms of energy consumption by decreasing the shock absorption function of muscles. This comes at the expense of the hard tissues (i.e., bones, joints, cartilage, etc), which must increase their shock absorbing function. This leads to a greater risk of injury and earlier onset of degradation injuries such as osteoarthritis.

It is desired to address or ameliorate one or more disadvantages or limitations associated with the prior art, or to at least provide a useful alternative.

### SUMMARY

In accordance with the present invention there is provided an apparatus for providing feedback to a user to optimise lower limb and core shock absorption performance, the apparatus including:
a sensory unit for collecting shock absorption data representing shock absorption by hard tissues of the user;
a data storage device;
a battery or recharging unit;
a communication unit configured to transmit the processed data to a user device; and
a housing unit for housing the sensory unit, the microprocessor unit, the data storage device, the battery or recharging unit and the communication unit, the housing unit configured to be fixed or attached to the user's body such that the sensory unit can collect the shock absorption data; and
a microprocessor unit coupled to the sensory unit, the microprocessor unit configured to:
   generate, by processing the shock absorption data, shock absorption performance data;
   store the shock absorption performance data to the data storage device; and
   transmit the shock absorption performance data to the user device via the communication unit,
wherein the shock absorption performance data can be used by a feedback module to generate and provide feedback to the user to improve the lower limb and core shock absorption of the user

In accordance with the present invention there is further provided a method for monitoring shock absorption performance of a user using an apparatus for providing feedback to a user to optimize lower limb and core shock absorption performance, the apparatus including a sensory unit, a microprocessor unit, a data storage device and a communications unit, the method including:
collecting, by the sensory unit, shock absorption data representing shock absorption by hard tissues of the user while the user is wearing the apparatus;
generating, by the microprocessor unit, shock absorption performance data by processing the collected shock absorption data; and
transmitting, by the communication unit, the shock absorption performance data to a user device, wherein the user device is configured to execute a feedback module that receives and interprets the transmitted shock absorption performance data in order to generate feedback that can be provided to optimize the shock absorption performance of the user when the user is walking or running.

In accordance with the present invention there is further provided a method for monitoring shock absorption performance of a user, the method including a user device executing a feedback module to:
receive shock absorption performance data transmitted to the user device;
generate feedback to be provided to the user to optimize the user's lower limb and core shock absorption, wherein generating the feedback is based at least in part on the received shock absorption data; and
providing the feedback to the user via a user interface of the user device.

In accordance with the present invention there is provided an apparatus for providing feedback to a user to optimise lower limb and core shock absorption performance, the apparatus including:
a sensory unit for collecting shock absorption data representing shock absorption by hard tissues;
a microprocessor unit coupled with the sensory unit for processing the collected data;
a data storage device for storing the processed data;
a battery or recharging unit;
a communication unit configured to transmit the processed data to a user device; and
a housing unit for housing the sensory unit, the microprocessor unit, the data storage device, the battery or recharging unit and the communication unit, the housing unit configured to be fixed or attached to the user's body such that the sensory unit can collect the shock absorption data,
wherein the processed shock absorption data can be used by a feedback module to generate and provide feedback to the user to improve the lower limb and core shock absorption of the user.

The sensory unit may include one or more of: an accelerometer, a gyroscope, a magnetometer, a temperature sensor, and a heart rate monitor.

The apparatus may include a display device configured to present a user interface, wherein the microprocessor is further coupled to the display device.

The data storage device may be part of the sensory unit or an external component such as a smart phone, tablet or other computing device.

In accordance with the present invention there is further provided a method for monitoring shock absorption performance of a user, the method including:
generating, by the above apparatus, shock absorption performance data, while the user is wearing the apparatus;
generating feedback that can be implemented by the user to improve lower limb and core shock absorption of the user, the generating based on the shock absorption performance data;
providing the feedback to the user via a user interface.

The feedback may include exercises to improve shock absorption performance over a period of time. The feedback may include recommendations or interventions to strengthen or loosen muscles or joints in the musculoskeletal system.

Providing the feedback to the user may include providing visual, auditory or tactile stimuli to the user.

The method may further include receiving, via the user interface, user data (including: height, weight, footwear, shock absorption parameters or previous performance levels, surface, location, etc). The generating of the feedback may be further based on the user data.

The method may further provide education and rehabilitation protocols for a user to self-manage an injury or complete an injury prevention or post-operation surgical rehabilitation protocol.

In particular, an embodiment provides a system including:
a wearable unit, for attaching to the medial ankle of a user, including a wireless transceiver, an accelerometer and an inertial measurement unit (IMU) to detect movement when walking and shock absorption by the ankle/lower limb of the user; and
a mobile device for communication with the wearable unit and including code of an application to execute at least some of the following:
a. access and display physio assessment questions for the user to answer;
b. process answers to the questions and display a series of objective movement tests for the user, such as squat and calf raise;
c. generate a user interface asking the user in response to the tests to mark on the user interface points of pain and score that pain;
d. generate and display instructions for the user to walk and obtain data from the wearable unit to generate user parameters relating to how the user moves when walking, such as shock absorption, knee data, step and speed;
e. analyse the user parameters obtained and generate recommendations for the user;
f. generate, based on analysis of the user parameters, coaching instructions to provide parameters for movement and a proper technique for walking, and generate and send commands to the wearable unit to guide the user, such as by haptics; and
g. generate weight loss instructions to guide the user to walk to produce optimal shock absorption to increase muscle activity (recruitment and contraction) and energy expended.

### BRIEF DESCRIPTION OF THE DRAWINGS

Embodiments of the present invention are herein further described, by way of non-limiting example only, with reference to the accompanying drawings, in which:

FIG. 1 is a schematic diagram of a user wearing an apparatus in accordance with an embodiment of the present invention, including an enlarged view of the apparatus;

FIG. 2 is a block diagram of a sensory unit of the apparatus in accordance with some embodiments of the present invention;

FIG. 3 is a flow diagram of a process performed by the apparatus;

FIG. 4 is a flow diagram of a process performed by a user device in accordance with some embodiments of the present invention;

FIG. 5 is a schematic diagram of a user device that is a computer;

FIGS. 6A and 6B are graphs showing data collected by the apparatus before and after feedback in accordance with an embodiment of the present invention;

FIGS. 7 and 8 are views of an embodiment of a housing unit and sensor unit of the apparatus;

FIGS. 9 and 10 are views of the senor unit with a charging device; and

FIGS. 11 to 13 are views of another embodiment of a housing unit and sensor unit of the apparatus.

### DETAILED DESCRIPTION

### Overview

Embodiments of the presently described apparatus and method relate to analysis of biomechanics and force transmission through the lower limbs of a person during ambulation or running, with a particular focus on the role of muscles to absorb force.

A person may perform the process of walking, running or other weight bearing activity with poor shock absorption for a number of reasons. For example, poor shock absorption can arise due to the person being unaware that they walk or run in a particular manner. It can also arise due to injury, muscle weakness, poor muscle activation, decreased flexibility or the person being post-operation. When a person performs walking, running or other weight bearing activity with poor shock absorption, their muscles (i.e., soft tissues) do not operate as effectively as they should, resulting in more force being transmitted through their bones or joints (i.e., hard tissues) or alternative soft tissues that will be overloaded or are not designed for this function. This can result in one or more of the following consequences:
(i) increased risk of injury for the person,
(ii) prolonged rehabilitation times or inability to rehabilitate to pre-injury level,
(iii) accelerated degradation of the hard tissues,
(iv) bony stress or joint dysfunction (in the athletic population), and
(v) degradation of the joint surfaces/cartilage.

Different individuals load different structures depending on their activity level, bio-mechanical variations and particular sports they are engaged in.

It may be desirable to provide an apparatus and method for providing feedback to mitigate or reduce such shock absorption inefficiencies. To this end, described herein is an apparatus and method that may allow for assessment of shock absorption performance, provision of usable feedback, provision of strategies or interventions to utilise the feedback effectively and alleviate or prevent pain/injury or early onset of musculoskeletal injury or osteoarthritis by optimising the shock absorption during walking and running.

Muscles perform an important function when a person is walking or running. They are well understood for their role in moving bones to enable physical movement. However, during walking and running, muscles have another role in absorbing vertical and horizontal forces acting on the body through controlled or active lengthening of the muscle. This process is known as an “eccentric contraction”, whereby the muscle acts as a tensile spring that lengthens as it absorbs force. This elastic energy is absorbed, not only through eccentric contraction of the muscles, but also through the tendons and connective tissue. This energy absorption in the soft tissues (muscles, tendons and connective tissue) minimises the amount of force transmitted through the hard tissues (bones, joints) or alternative soft tissues that are not designed for this function.

Phases of the gait cycle include a foot strike. The foot strike is generally in the form of a heel strike when walking, and a midfoot to forefoot strike when jogging or running. The faster a person's pace, the further the strike point is generally located up the foot (i.e., towards the toes). In other words, a heel strike is expected for walking, while a midfoot strike and a forefoot strike are respectively expected for jogging and sprint running.

At each foot strike, a combined force acts on a person's body. In the sagittal plane (running from the person's head to foot, and creating two equal halves of the body—left and right), the resultant force vector can be broken down into vertical and horizontal components: horizontal component is described as the braking or accelerating force (depending on the strike position relative to the person's centre of gravity), and the vertical component is described as the ground reaction force. These two forces can be minimised by making changes (i.e., “optimising”) the gait of person to decrease excessive force applied to the person's load bearing structures (i.e., hard tissues) and thereby decreasing the risk of injury. This can also be performed for the frontal and transverse planes.

In the context of walking, transmission of force from a heel strike takes the following path through the skeletal system:
(i) from the calcaneum to the talus (leg path) and cuboid (foot path);
(ii) the cuboid (midfoot bone) and a portion of the talus (midfoot bone) distribute force the tarsal bones, which spread and thus absorb force through the foot;
(iii) the talus transfers force upwards through the ankle joint to tibia (80% of the force) and fibula (20% of the force);
(iv) the tibia then transfers this force up through the knee joint to the femur, and the femur transfers this force up through the hip joint to the pelvis;
(v) the pelvis acts as a force distributor, transferring the force around the pelvic bones (ischium, pubis and ilium) and transferring this dampened force to the spine via the sacrum;
(vi) at this point, the upper body weight force is being transmitted down the spine, through the sacrum and into the pelvis.

The muscles and biomechanics involved in shock absorption are proficient. On landing or foot placement, the foot/ankle/calf complex absorbs 60% of the force being transmitted up the lower limbs. Running generates more force for shock absorption compared with walking—the force may be up to three times the body weight of the runner at each step (and even more in an inefficient runner).

Shock absorption of the ankle joint is an example through which the process of energy absorption can be examined. As the runner strikes the ground with the midfoot or forefoot, the ankle and foot are almost pointed, or are in degrees of plantar flexion (depending on the speed and technique of the runner). As the runner lands and absorbs force, the ankle moves from ankle plantar flexion to ankle dorsiflexion, while the calf muscle eccentrically (lengthening) contracts and the achilles tendon and connective tissue elongate. This creates an elastic stretch while absorbing force—similarly to a lengthening spring. Thus, the muscular system can actively absorb the forces, consequently reducing the load transmitted through the hard tissues (i.e., bones and joints) and further up the kinetic chain to the knees, hips, pelvis and spine.

The muscular system has the capabilities to perform this shock absorption function, relying on the skeletal system to have sufficient range of movement in the joints to allow the shock absorption to occur over a longer time interval. In the case of an ankle, if a person does not have enough dorsiflexion range of movement at the ankle joint, then as the ankle dorsiflexes to allow the muscles to shock absorb, the movement is blocked and the ability of the muscles to absorb force ceases—thus the shock absorption process is compromised. Compromise of the shock absorption process can also occur in a person with injury in the limb (on the same side as the ankle) that causes decreased range of movement, or a person that walks or runs with a poor technique that may need to be re-educated or modified.

In turn, the neurological system (brain and nerves) works in conjunction with the muscular system to appropriately coordinate the timing and degree of contraction of the muscles—these systems work together as a neuromuscular unit.

The apparatus and method described herein look to optimise the working of the neuromuscular unit to thereby improve the shock absorption of the soft tissues during walking and running.

As mentioned above, if the force associated with a foot strike is not absorbed in the soft tissues, then it is transmitted through the hard tissues. Transmission through the hard tissues can be detected as increased displacement of the body—generally more noticeable in the lower limbs—that may be described as a ‘jolt’. This jolt (i.e., poor/undesirable shock absorption) can be detected by a sensor on the body of the user (e.g., on the user's tibia, fibula, hip, chest, spine or head, etc.), and feedback based on this detection can be provided to the user to optimise their neuromuscular system's ability to absorb forces. Feedback may also be provided to the user through referral to an appropriate health professional or a self-guided rehabilitation program.

In accordance with the apparatus and method described herein, by placing a sensor on the body of a user, shock absorption by hard tissues of the user can be detected. Feedback based on the detected shock absorption can be generated and provided to the user to optimise the shock absorption function of their lower limb and core muscles. This may:
1) negate or prevent pain associated with poor shock absorption of ambulation/gait and running or activities (e.g.—sporting activity, training drills, rehabilitation exercises, etc); and/or
2) provide feedback on the efficiency of their skill performance (activated or mindful or ‘light’ walking technique), gait and shock absorption performance with or without a software program that will either provide immediate feedback to enhance the user's performance in real time or provide an overview of performance at a later date/post performance.

The feedback can include instructions or advice on appropriate gait techniques and targeted mobilisation of joints or strengthening of muscles. Such instructions or advice can be specific to the user, or generalised to a particular injury, functional requirement (e.g., weight loss) or patient group.

In addition, individuals suffering from osteoporosis may want to maintain or increase their bone density by being heavier on their joints. This may be achieved by taking at least a specific number of steps per day with a set amount of load going through the bone, and/or decreasing muscle activity while walking. For example, this may be useful in members of the younger population who suffer from eating disorders that might affect their long-term bone health. The apparatus and method described herein may be used for assessment of shock absorption performance as a measure of muscle recruitment, provision of usable feedback, strategies or interventions to maintain or improve bone density by decreasing muscle recruitment and increasing shock absorption through the hard tissues during walking or running.

The shock absorption data collected by the apparatus can also be collated for research purposes, to provide feedback to the user, a coach, a medical/health professional or a scientist, to improve function, prevent pain and manage a program.

### Apparatus

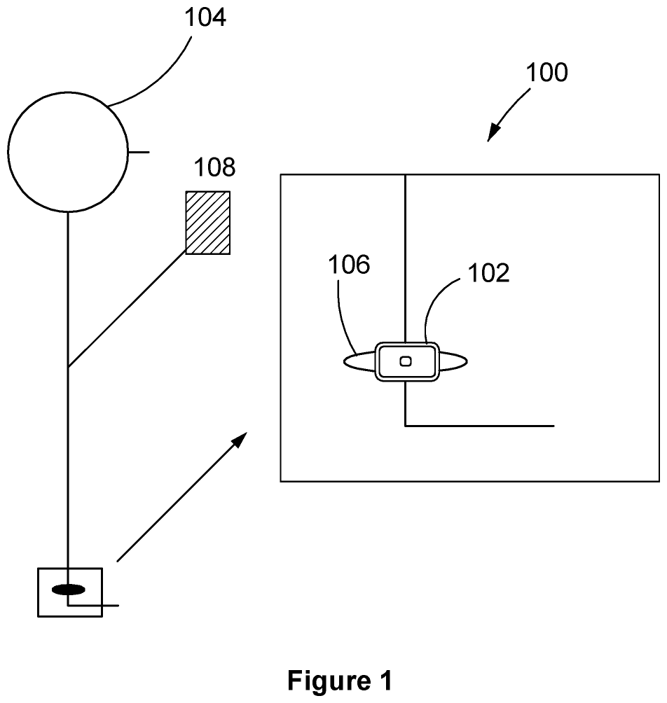

As shown in FIG. 1, an apparatus 100 in accordance with an embodiment of the present invention includes a sensory unit 102 to be placed on the body of a user 104. The sensory unit 102 is for collecting shock absorption data representing shock absorption of hard tissues of the user 104. In the embodiment shown, the sensory unit is placed on the shinbone (tibia) of the user 104. In other embodiments, the sensory unit 102 may be placed on other locations of the body of the user 104, such as the chest, arm, neck or hip. The sensory unit 102 may be located on the shinbone due to the ability to obtain reliable measurements in this position on the body of the user 104. The sensory unit 102 is held in place on the body of the user 104 by a housing unit 106, such that the sensory unit 102 is secured directly against skin overlying the tibia bone. This provides an accurate measure of the shock absorption by the hard tissues, e.g., force being transmitted through the hard tissues. The housing unit 106 houses the sensory unit, the microprocessor unit, the battery or recharging unit and the communication unit.

The apparatus 100 includes a microprocessor unit 204, a data storage device 206, a battery 210 or recharging unit, and a communication unit 208 (not shown in FIG. 1).

In some embodiments, the apparatus 100 includes a display device configured to present a user interface, wherein the microprocessor unit 204 is further coupled to the display device.

The microprocessor unit 204 receives the collected shock absorption data from the sensory unit 102 and processes the collected data such that:
(a) the processed data can be stored at the data storage device 206; and/or
(b) the communication unit 208 can transmit the processed data to a user device 108.

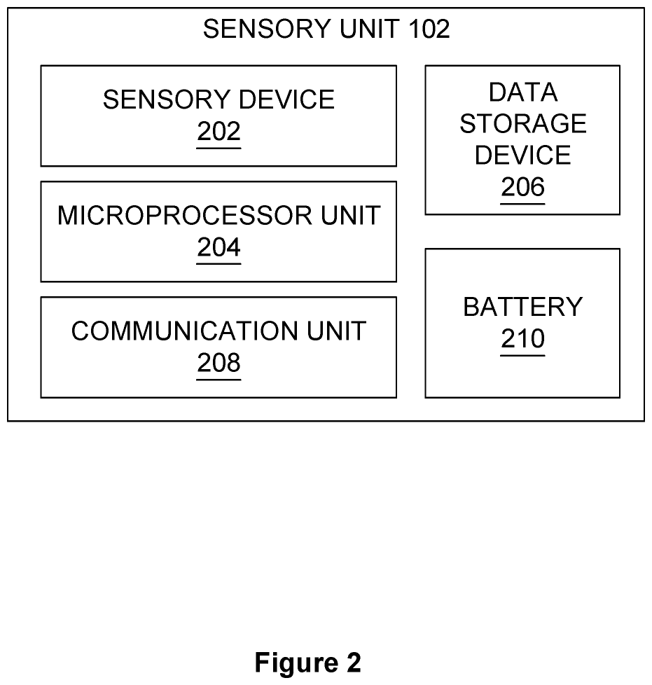

As shown in FIG. 2, the sensory unit 102 includes a sensory device 202. The sensory device 202 may include, but is not limited to: an accelerometer, a magnetometer, a gyroscope, an altimeter, strain gauges, a pressure sensor or gauge, a global positioning system, a proximity sensor, and a piezoelectric sensor.

The sensory unit 102 can also include the microprocessor unit 204 and/or the data storage device 206, the communication unit 208 and the battery 210. However, in some embodiments one or more of these components may be located outside the sensory unit 102.

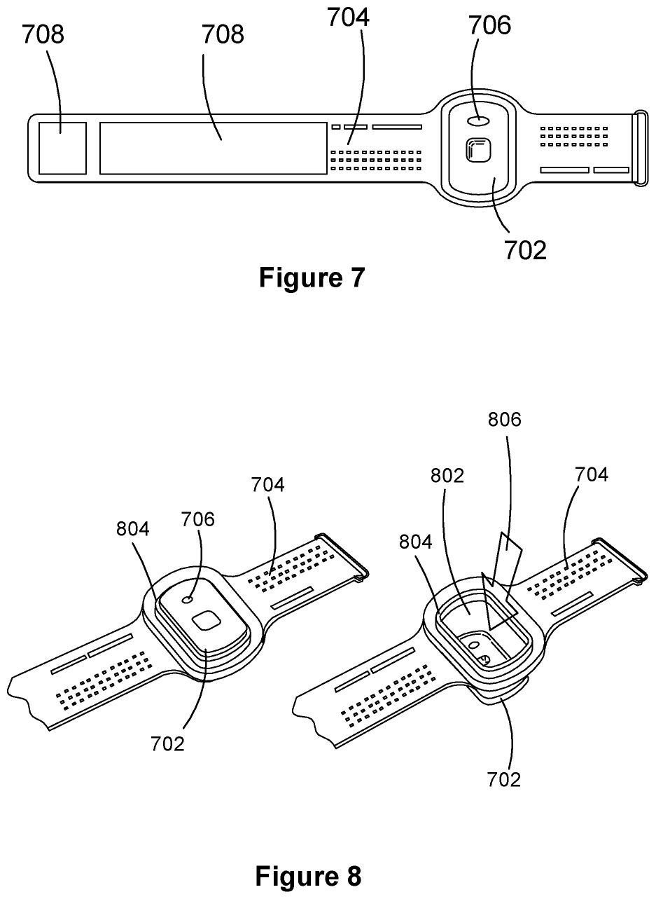

As shown in FIG. 7, the housing unit 106 can include a casing 702 and a band 704.

The casing 702 encloses the sensory unit 102, the microprocessor unit 204, the data storage device 206, the battery 210 and the communication unit 208. The casing 702 may have a light source 706 such as an LED. The light source 706 may indicate a power status of the apparatus 100 and/or feedback based on the processed data. For example, the light source 706 may be green (and optionally flashing) to indicate that the apparatus 100 is powered ‘on’, the light source 706 may be blue to indicate that the apparatus 100 is charging, and/or the light source 706 may be red (and optionally flashing) to indicate to the user 104, based on the processed data, that the user's shock absorption performance does not fall within predetermined parameters. Although described in relation to the casing 702, the light source 706 may be otherwise provided on the apparatus 100 such that it can be observed by the user 104, such as on the band 704.

The casing 702 may be made of a hard plastic material.

The band 704 allows the casing 702, and thereby the sensory unit 102, to be secured on the body of the user 104. As shown, this can be achieved by the band 704 including one or more fasteners 708, such as hook and loop fasteners. The band 704 is made of a flexible material so that it is capable of being adapted to the body of the user 104, e.g., around a limb of the user 104. For example, the band 704 may be made of fabric (e.g., including elastane), silicon or rubber.

As shown in FIG. 8, the casing 702 and the band 704 are configured such that the casing 702 can be removably attached to the band 704. The band 704 includes an aperture 802 defined by a surround 804 in which the casing 702 can be securely fitted. The user 104 can apply pressure in a downwards direction 806 in order to remove the casing 702 from the band 704, e.g., for charging the battery 210.

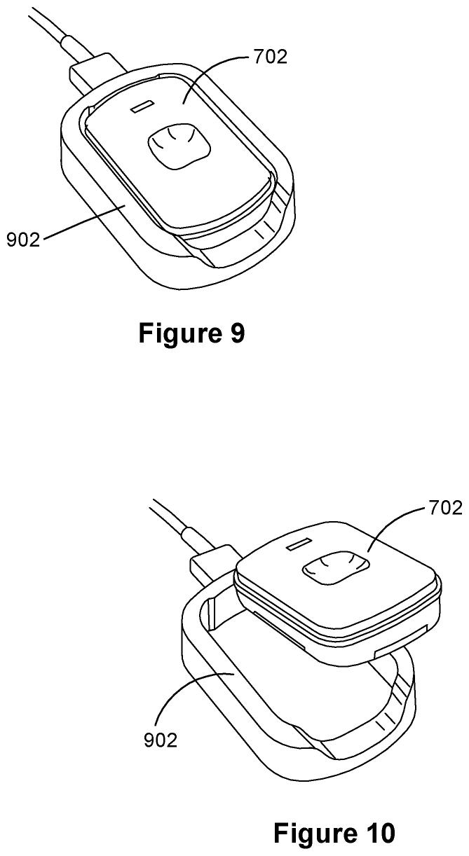

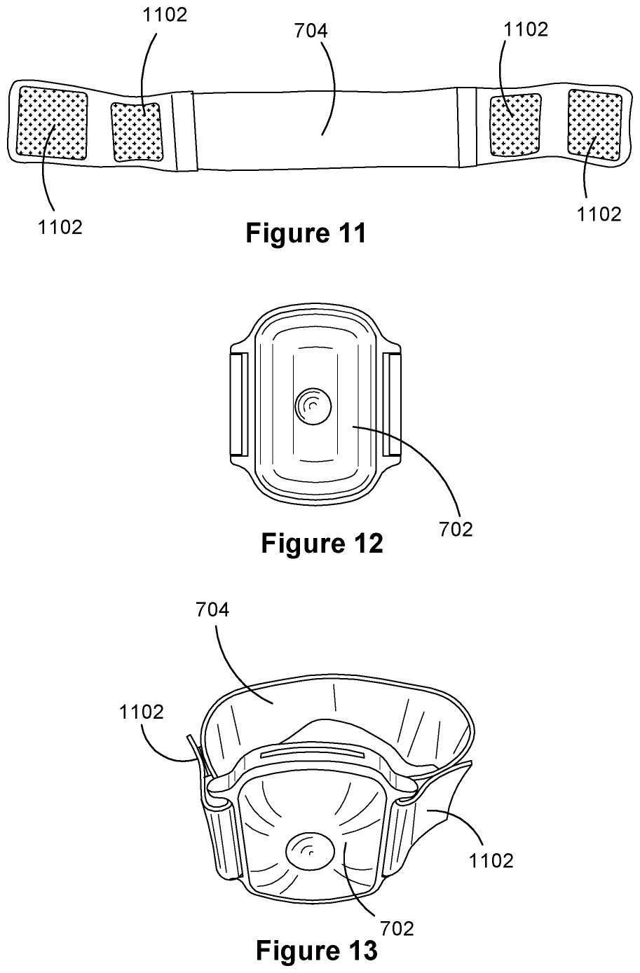

As shown in FIGS. 11 to 13, in another embodiment the band 704 is configured to be removably attached to the casing 702 by hook and loop fasteners 1102. This may allow the band to be adjustable such that the apparatus 100 can be securely placed on different parts of the body (e.g., shin, chest, hip, arm) on users of various sizes. In particular, the band 704 allows the casing 702 to have primary contact on and flush against the bone of a user, with de minimis pressure on the skin and without compressing a tendon, i.e. the Achilles tendon. This allows the sensory unit 102 to be kept on the bone all day without detrimental pressure or irritation for a user.

As shown in FIGS. 9 and 10, once the casing 702 is removed from the band 704, the battery 210 can be charged by placing the casing 702 in a charging device 902.

### Method

A method for monitoring shock absorption performance of a user according to embodiments of the present invention includes:
generating, by the above apparatus, shock absorption performance data, while the user is wearing the apparatus;
generating feedback that can be implemented by the user to improve lower limb and core shock absorption of the user, the generating based on the shock absorption performance data;
providing the feedback to the user via a user interface.

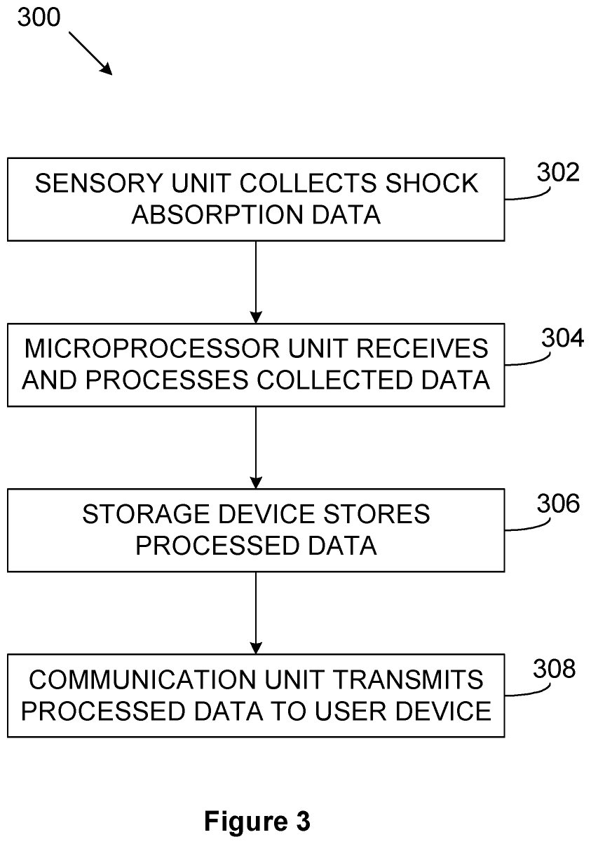

FIG. 3 is a flow diagram of a method 300 performed by an apparatus 100 to generate shock absorption performance data.

At step 302, the sensory unit 102 collects the shock absorption data representing shock absorption of the hard tissues of the user 104 using the sensory device 202. For example, where the sensory device 202 includes an accelerometer, the sensory unit 102 may collect shock absorption data including acceleration data which represents the shock absorption of the hard tissues of the user 104. The shock absorption data also represents or is indicative of muscle recruitment, energy consumption and fat burning.

At step 304, the microprocessor unit 204 receives and processes the collected shock absorption data. The processing may include analysing, refining and preparing the collected data for communication by the communication unit 208. For example, the microprocessor unit 204 may remove any ‘noise’ and/or vibration feedback from the collected data. The processed data is described as “shock absorption performance data”, and can be used by a feedback module to generate and provide feedback to the user to optimize their lower limb and core shock absorption, as described below. The feedback is data generated by the feedback module.

The microprocessor unit 204 can identify that the shock absorption performance data is outside (e.g., above or below) one or more predetermined thresholds and accordingly cause the sensory unit 102 to generate a signal to the user 104 to inform them that they are outside of the predetermined thresholds. The signal can be, for example, a visual signal such as activation of a light (e.g., light source 706), an audible signal such as activation of an alarm sound, a tactile signal such as a vibration that the user 104 can sense. The signal may be generated at least in part by the sensory unit 102. The signal may be generated at least in part by the user device 108 (e.g., a smart phone). The predetermined thresholds may be based on one or more of the user details and/or the user parameters received at steps 404 and 406, discussed below, and received by the microprocessor unit 204 via the communication unit 208. The signal is for prompting the user 104 to alter their behaviour so that the shock absorption performance data ceases to be outside the predetermined thresholds and the lower limb and core shock absorption of the user 104 may be improved.

At step 306, the data storage device 206 stores the processed data. The stored data is able to be later transferred to another computing device such as the user device 108.

At step 308, the communication unit 208 transmits the processed data to the user device 108. This transmitting can be “live”, i.e., in real time as data is collected by the sensory device 202 and subsequently processed by the microprocessor unit 204. Alternatively, the communication unit 208 may transmit the processed data (e.g., as stored in the data storage device 206) to the user device 108 after a delay. The communication unit 208 transmits the processed data to the user device 108 via a wired or wireless communication link such as bluetooth, wi-fi, USB, fibre optic cable, infrared light, ZigBee, etc.

Additionally or alternatively to being stored locally on the data storage device 206 of the apparatus 100, the processed data can be stored by the user device 108—locally on the user device 108 or remotely on a server, including a remote cloud server.

### User Device and Feedback Module

The user device 108 may be, for example, a computer, smart phone, tablet, smart watch, etc. The user device 108 is configured to execute a feedback module that receives and interprets the data transmitted to the user device 108 from the communication unit 208 of the apparatus 100 in order to generate and provide the user 104 with feedback to optimize the shock absorption performance of the user when the user is walking or running. Feedback is data generated by the feedback module as described herein.

The feedback module is configured to receive user data provided by the user 104, e.g., at the user interface of the user device 108.

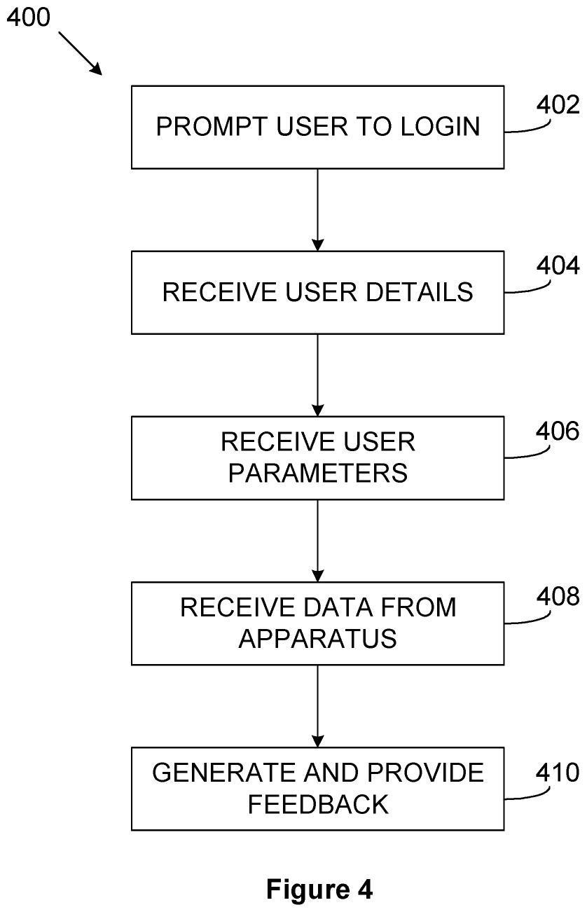

FIG. 4 depicts a flow diagram of a method 400 performed by the feedback module at the user device 108 in accordance with some embodiments of the present invention.

At step 402, the feedback module prompts the user 104 to provide login details via the user interface.

At step 404 the feedback module receives, via the user interface, user details input by the user 104. The user details may relate to one or more of the following:
(i) BMI;
(ii) Height;
(iii) Weight;
(iv) Leg length;
(v) Ankle circumference;
(vi) Ankle tissue type, e.g., bony, oedematous (swollen), adipose (fat tissue);
(vii) Shoe/footwear type (e.g., bare feet, runners);
(viii) Activity Surface (e.g., sealed road, grass, gravel, concrete, floorboards);
(ix) Injury;
(x) Operations or other medical treatments;
(xi) Previous performance levels, recorded using the apparatus or otherwise determined, including any of the following:
   a. Pre-Injury or Operation Performance Levels;
   b. Full Recovery Performance Level;
   c. Surface Specific Performance Level;
   d. Footwear Specific Performance Level;
(xii) Ambulatory or Running Goals including any of the following:
   a. walking/running speed goals,
   b. walking/running distance goals,
   c. ‘change of direction’ goals, e.g., cutting, stepping, figure-eights;
(xiii) Weight loss goals;
(xiv) Distance travelled (e.g., 100 m, 800 m, 5 km, 20 km);
(xv) Duration;
(xvi) Terrain;
(xvii) Limits of pressure or shock absorption performance (upper or lower limits);
(xviii) Cumulative Shock Absorb in a Day;
(xix) Overall Daily Performance;
(xx) Cumulative Body Load or Shock Transmitted (by step, session or day, etc).

At step 406, the feedback module receives, via the user interface, user parameters input by the user 104. The user parameters may include user objectives, user workouts and/or user environments. The user parameters may include units of g (i.e., measures of acceleration). The user parameters may include percentages/units of performance. The user parameters may include minimum and maximum heart rates. The feedback may be based in part on such user parameters. The feedback module may have one or more default parameters applied in the absence of parameters being input by the user 104.

If parameters have been received or otherwise applied at step 406, the feedback module may determine one or more thresholds for the shock absorption performance data generated by the apparatus described herein. The thresholds define a desirable range of shock absorption performance data that may allow the user to achieve one or more outcomes such as recovery/rehabilitation from injury, weight loss, increased speed, bone density maintenance, etc. The method can include the apparatus identifying that the generated shock absorption performance data is outside the determined thresholds, and generating a signal to alert the user 104 that the shock absorption performance data is outside the thresholds and thereby prompt them to alter their behaviour so that the shock absorption performance data ceases to be outside the predetermined thresholds.

After the user 104 has performed an activity (such as walking or running) while wearing the apparatus 100, at step 408, the feedback module receives the processed data transmitted to the user device 108 by the communications unit 208.

At step 410, the feedback module generates and provides feedback to the user 104 to optimise the user's lower limb and core shock absorption during a current movement or exercise and/or for future movement or exercise. For example, the feedback may be for the user 104 to alter a current movement or exercise being performed by the user 104, or to modify an existing stored program or exercise protocol of the user 104. The generation of the feedback is based at least in part on the data received at step 408. The generation of the feedback may be further based on the received user data, i.e., the user details received at step 404 and/or the user parameters received at step 406. The feedback is provided to the user 104 via the user interface.

The generation of the feedback may include analyzing the received data to identify data that represents poor shock absorption by the user 104, i.e., excessive shock absorption by the user's hard tissues. For general issues, this may result in a predetermined feedback response. For other more user-specific issues, coaching staff or health professional guidance may result in user-specific feedback to the identified poor shock absorption data.

The feedback can include strengthening exercises, in particular, where the user 104 is consistently creating poor shock absorption strategies or analysis of a health professional a specific or targeted exercise routine may be entered into the feedback module. For example, foot intrinsic muscles may be targeted as weakness or contributing factor to poor shock absorption on a particular terrain, type of exercise or duration of exercise. The exercises can be displayed on the user interface, and any modifications to these exercises can also be input via the user interface. The display of each exercise can include displaying one or more of:
(a) a description of the exercise,
(b) a picture or video of the exercise, and
(c) an indication of the duration, repetitions, intensity and/or resistance of the exercise.

The feedback may be generated and provided to the user 104 “live” (i.e., in real time) for immediate intervention via the user interface or at the sensor level. However, provision of the feedback can also be delayed by storing the feedback for later review by the user 104 or other professionals such as sports scientists, sports medicine professionals or coaching staff.

This data storing can occur at the said sensory data storage level or at the software interface level of the method and system. Thus, the user may be able to use the apparatus with or without the user device 108. This may be convenient for a user 104 who is using the apparatus while going on a run, and does not wish to carry the user device 108 (such as a smart phone) with them during the run.

The generated feedback may provide the user 104 with direct information on their shock absorption performance in one or more of the following respects:
(i) step by step feedback (i.e., feedback that is ‘per step’ or step specific);
(ii) feedback for multiple steps (the multiple steps can be grouped by location, distance or time period);
(iii) location of performance;
(iv) direction;
(v) velocity;
(vi) force;
(vii) strike zone;
(viii) quality of performance;
(ix) review of performance on user's entered target parameters.

The stored user feedback enables review of the shock absorption performance, may include specific analysis or comparisons between:
(i) individual steps;
(ii) left side versus right side;
(iii) groups of multiple steps, or all steps in an overall session;
(iv) location;
(v) direction;
(vi) velocity;
(vii) previous performances.

When provided to the user 104, the generated feedback may enable the user 104 to improve or optimise their shock absorption performance as an immediate intervention by prompting or guiding a better technique.

This prompting or guiding may be made by the feedback module or over-ridden by the user 104 or external party to provide any additional recommendations from a sports science, sports medicine health professional or coaching staff.

The feedback may prompt or instruct the user 104 to perform specific movement alterations, and/or give generic overview instructions or alerts. For example, the feedback may include one or more of the following prompts, instructions or alerts:
(i) lift right knee higher;
(ii) strike with heel;
(iii) strike with heel;
(iv) softer knees;
(v) roll onto forefoot;
(vi) softer strike with heel;
(vii) more forefoot contact;
(viii) lightly place heel;
(ix) walk with your natural walking style;
(x) faster;
(xi) slower;
(xii) increase left foot intrinsic muscle contraction;
(xiii) walk lightly;
(xiv) fatigue is evident.

The data received at step 408 may be used to trigger one or more other processes carried out feedback module, such as providing coaching, sports medicine feedback or sports science feedback or prompting the user 104 to refer to sports/medical professionals to seek advice. Utilising wireless communications (Bluetooth, WiFi, Zigbee, 5G, GPS, etc.), a whole team of players (e.g. football, soccer, rugby) would be able to be monitored during training and game play.

### Example 1: Improving Shock Absorption Efficiency

The feedback module is able to identify, based on the received data, a strike zone of the user 104. The identified strike zone may be one of: heel, midfoot or forefoot. The feedback module can then determine whether the identified strike zone is appropriate or optimal for one or more user details input by the user (at step 404), such as the type of gait, footwear, terrain, etc. For example, the feedback module may identify the strike zone of the user as being a forefoot strike when the user details indicate that the user is walking. The feedback module can then subsequently determine that this is an inappropriate strike zone for walking and generate feedback data prompting the user to perform heel striking.

### Example 2: Weight Loss

Where the user data includes a goal relating to weight loss, the feedback module identifies, based on the received data, that the user 104 is not performing an exercise (such as walking or running) with a technique requiring increased muscle activity and thus increased energy consumption. The feedback module then generates feedback data to prompt the user to adopt the technique requiring increased muscle activity. This results in the user increasing their energy consumption and thereby losing weight (when this technique is maintained over time).

The thresholds for the shock absorption performance data can include an upper threshold. For example, where the shock absorption performance data includes acceleration data, the upper threshold can be a maximum acceleration. The maximum acceleration can be at least 1 g. The maximum acceleration can be no more than 2 g.

At step 410, the feedback module generates and provide feedback data to the user 104 if the shock absorption data is above the upper threshold. In other words, feedback data is generated and provided to the user 104 if the user needs to walk ‘lighter’ (i.e., with more efficient shock absorption, requiring more muscle activity and thereby more energy consumption and fat burning). The feedback data can generate a signal to the user 104 (such as a visual, audible and/or tactile signal) which may be provided at least in part by the sensory unit 102 and/or the user interface. The feedback data can include instructions to the user to walk more lightly, e.g., including one or more of the following: to lightly place their heel or foot, to not stomp, to walk taller, to not smash their heel to the ground.

If the shock absorption data is below the upper threshold, this may suggest that the shock absorption performance is sufficient to give rise to energy consumption that will result in weight loss. As such, no feedback data may be generated. Alternatively, feedback data may be generated and provided to the user 104 to inform the user 104 of their good technique/performance (during and/or after the performance of the activity), i.e., to provide positive reinforcement to the user 104. However, other feedback data may be generated and provided to the user 104 if the shock absorption data indicates poor walking technique. For example, the feedback data can generate one or more of the following instructions on the user interface as appropriate: soften the knees, do not strike with the midfoot or forefoot, lightly place the heel, walk lightly and quietly.

The feedback module may generate and provide the user 104 with a summary of their light walking performance, energy consumption and/or weight loss performance. The feedback module generates a comparison between the user's ‘normal’ (i.e., pre-feedback) walking performance and the user's walking performance as modified by the feedback data. The summary and/or comparison may be generated at least partially based on the shock performance data. The summary and/or comparison may be generated at least partially based on the user details.

A trial was performed for users attempting to lose weight. It was found that for equal conditions, distance and amount of time walking, participants generated at least between 15-30% more energy consumption when walking informed by feedback, as described above, compared with no feedback.

The feedback module can also access and provide, in combination or as separate components, advice on nutrition, mental health and injury prevention to assist the user.

### Example 3: Osteoporosis and Bone Density

As discussed hereinbefore, the user 104 may wish to improve their bone density by decreasing their muscle activity when walking to increase shock absorption in their hard tissues. Hence, the thresholds for the shock absorption performance data can include a lower threshold. At step 410, the feedback module may generate and provide feedback to the user 104 if the shock absorption data is below the lower threshold. In other words, feedback is generated and provided to the user 104 if the user needs is walking too lightly to maintain or improve their bone density. The feedback may include instructions to the user to walk more heavily, e.g., to sink into the ground when walking, to strike more heavily with the foot and/or to generate more steps with a higher load force. The feedback may include instruction to the user to relax as they walk.

### Example 4: Rehabilitation

The user 104 may use the apparatus and methods described herein to rehabilitate an injury. The user details received by the feedback module at step 404 can include, for example, one or more of: an injury type, an injured body region, symptoms, pain type (e.g., sharp or dull), pain level, hours of sleep.

At step 410, the feedback module may generate and provide feedback to the user 104 based on the user details. For example, the feedback module may generate and provide the user 104 with instructions to perform exercises based at least in part on the injury type. The feedback module may receive, via the user interface, further user details input by the user 104 after performing each exercise. For example, the further user details can include one or more of the following: pain level, perceived rate of exertion, ease of performance (e.g., scored out of 10 or 100).

The feedback module may generate and provide further feedback to the user based on the user details and the further user details. For example, if the further user details indicate that the user 104 is pain-free after performing an earlier exercise, the feedback module may generate feedback instructing a different exercise, i.e., allowing the user 104 to progress to a next step of their rehabilitation.

The feedback module is able to operate on any or all of the following data:
1. patient profile/user details;
2. subjective examination data representing questions answered that day or previously (eg—sleep, wellness questions, pain in activities of daily living, pain with exercises);
3. objective test performance data representing pain, perceived rate of difficulty, etc. performed that day or previously;
4. gait analysis or performance analysis data representing performance of shock absorption in gait, running or other tests/performances; and
5. exercise, management or rehabilitation adherence data representing how diligent the user has been with rehabilitation exercises and management over a previous time period.

The shock absorption performance data generated by the apparatus, while the user is wearing the apparatus, can be used to generate feedback in relation to the user's performance of the exercises and thereby improve the user's recovery from the injury. The shock absorption performance data may also be used to monitor compliance of the user 104 with respect to performing the exercises instructed by the feedback module. I.e., the shock absorption performance data can be used to identify if the user has failed to perform the exercises, failed to perform the instructed number of repetitions, or failed to perform a complete number of sets of repetitions.

### Knee Osteoarthritis Trial

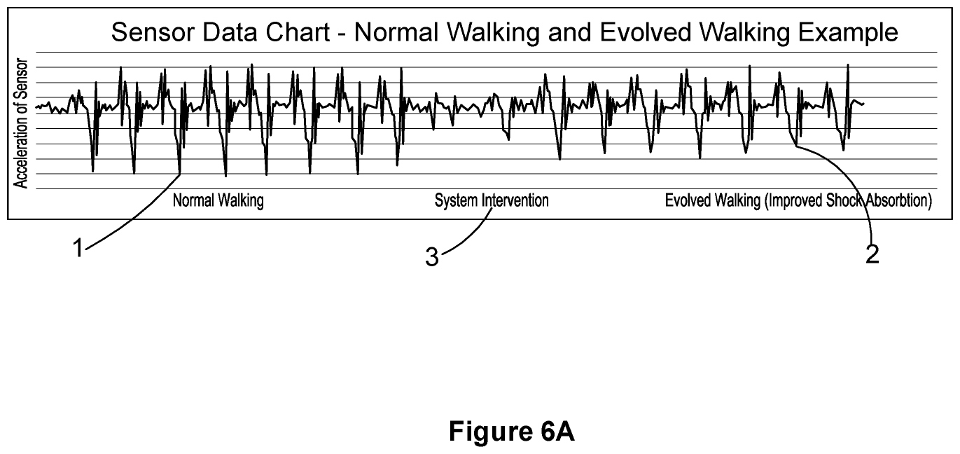

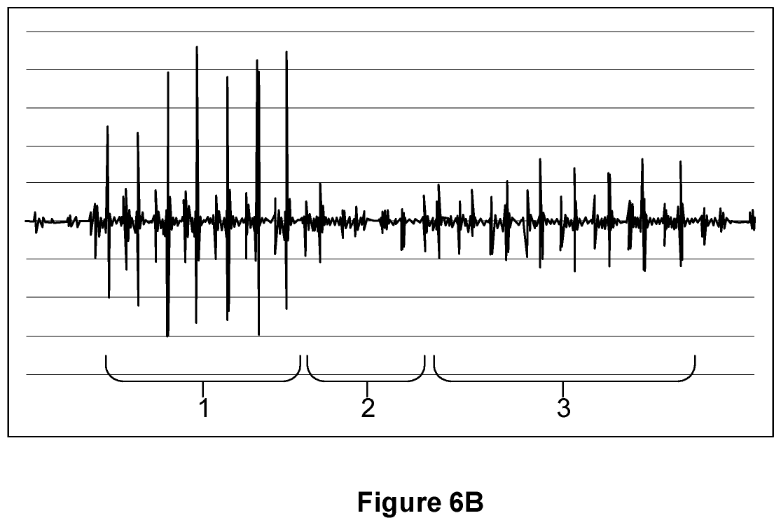

A six week trial was performed for users (patients) with knee osteoarthritis. Data collected from a first patient in the trial is shown in FIG. 6A, where the x-axis represents time and the y-axis represents acceleration detected by the sensory device 202 (indicating shock absorption). Data collected from a second patient in the trial is shown in FIG. 6B, where the x-axis represents time and the y-axis represents acceleration detected by the sensory device 202 (indicating shock absorption).

The shock absorption performance of this patient before any feedback is shown in section 1. The shock absorption performance of this patient during the provision of feedback generated by the feedback module is shown in section 3. The shock absorption performance of this patient following the provision of the feedback is shown in section 2.

By relying on the apparatus 100 and methods described herein to facilitate gait re-education and osteoarthritis management, it was found that users may reduce their pain by up to 91% and improve function on average by 38%. Therefore, use of the apparatus to re-educate gait may decrease a user's pain, improve the user's function, decrease the need for medications, improves the user's confidence in knee, decrease any need for surgery and improve the user's quality of life.

### User Device

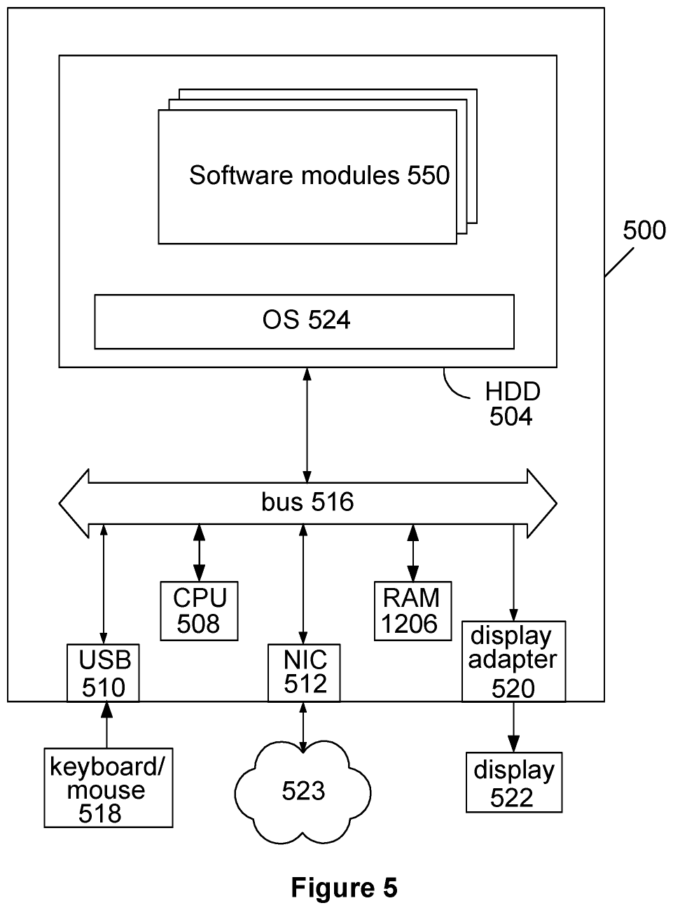

The method of optimising shock absorption during walking or running in a person is implemented at least partly on the user device 108. The user device can be a computer 500, as shown in FIG. 5.

The computer 500 may be based on a standard computer, such as a 32 or 64 bit Intel architecture computer produced by Lenovo Corporation, IBM Corporation or Apple Inc. The data processes executed by the computer 500 are defined and controlled by computer program instruction code and data of software components or modules 550 (including the feedback module) stored on non-volatile (e.g.—hard disk) storage 504 of the computer 500. The processes performed by the modules 550 can, alternatively, be performed by firmware stored in read only memory (RAM) or at least in part by dedicated hardware circuits of the computer 500, such as application specific integrated circuits (ASICs) and/or field programmable gate arrays (FPGAS).

The computer 500 includes random access memory (RAM) 506, at least one microprocessor 508, and external interfaces 510, 512, 514 that are all connected by a system bus 516. The external interfaces include universal serial bus (USB) interfaces, a network interface connector (NIC) 512, and a display adaptor 514. The USB interfaces 510 are connected to input/output devices, such as keyboard and mouse 518. The display adaptor 514 is connected to a display device, such as an LCD display screen 522. The NIC 512 enables the computer 500 to connect to a communications network 523. The network 523 may include one or a combination of existing fields, such as a LAN, WAN, the PSTN, the internet, mobile cellular telephone networks, etc. The computer 500 includes an operating system (OS) 524, such as Microsoft Windows, Mac OS X or Linux. If the computer 500 is a hand held or worn device, the OS 524 may be IOS, Android or WatchOS. The modules 550 all run on the OS 524, and include program code written using languages, such as C, Ruby or C #.

In one example, the computer 500 is a server computer to which a client computer may connect over the network 510. In this implementation, client software modules running on the client computer interact with software modules 550 running on the server computer 500. The client software modules may include compiled executable code configured to run on the OS 524, or may be configured to run within the web browser on the client computer for example.

The modules 550 may include code for monitoring shock absorption data values received at step 406 and providing feedback of an individual's shock absorption performance based on those values. The monitoring may be based on data accumulated by the sensory unit, as described above. The modules 550 may also include a module that allows for upload of data from other wearable devices such as heart rate monitors and the like.

The user may or may not login, as shown in FIG. 4, depending on their individual set up. User details maybe or may not be entered. Parameters, either actively sensed for the variables of that session (e.g.—terrain, footwear, activity, etc.), entered by the user or default settings may also be entered. The activity will be performed and with the parameters for that session will provide the constraints or resultant feedback to the user if outside these set variable perimeters. Stored data from a session can be viewed on an apparatus with an appropriate interface.

Although the feedback module has been described as being executed on the user device 108, in some embodiments the feedback module may be executed by the microprocessor unit 202 of the apparatus 100 such that the apparatus 100 can be used as a standalone device.

The apparatus and methods described herein may be used in a clinic with a therapist or other health professional, in a telehealth setting with a therapist or other health professional, or in an in person or virtual group class with a health or fitness professional. The apparatus and method can also be used by the user independently while in a private/home environment or at a gym.

Many modifications will be apparent to those skilled in the art without departing from the scope of the present invention.

The reference to any prior art in this specification is not, and should not be taken as, an acknowledgement or any form of suggestion that the prior art forms part of the common and general knowledge in the field of endeavour to which this specification relates.

1. An apparatus for providing feedback to a user to optimise lower limb and core shock absorption performance, the apparatus including:
a sensory unit for collecting shock absorption data representing shock absorption by hard tissues of the user;
a data storage device;
a battery or recharging unit;
a communication unit configured to transmit the processed data to a user device; and
a housing unit for housing the sensory unit, the microprocessor unit, the data storage device, the battery or recharging unit and the communication unit, the housing unit configured to be fixed or attached to the user's body such that the sensory unit can collect the shock absorption data; and
a microprocessor unit coupled to the sensory unit, the microprocessor unit configured to:
generate, by processing the shock absorption data, shock absorption performance data;
store the shock absorption performance data to the data storage device; and
transmit the shock absorption performance data to the user device via the communication unit,
wherein the shock absorption performance data can be used by a feedback module to generate and provide feedback to the user to improve the lower limb and core shock absorption of the user.

2. The apparatus of claim 1, wherein the housing unit is configured to be fixed or attached to the user's shinbone such that the sensory unit can collect the shock absorption data.

3. The apparatus of claim 1 or 2, wherein the housing unit includes a casing and a band, the casing enclosing the sensory unit and the band being configured to secure the casing to the user's body such that the sensory unit can collect the shock absorption data.

4. The apparatus of claim 3, wherein the casing and the band are configured such that the casing can be removably attached to the band.

5. The apparatus of claim 4, wherein the band includes an aperture in which the casing can be securely fitted to removably attach the casing to the band.

6. The apparatus of any one of the preceding claims, wherein housing unit includes a light source, the apparatus being configured such that the light source can indicate at least one of:
a power status of the apparatus; and
the feedback to the user to improve the lower limb and core shock absorption of the user.

7. The apparatus of any one of the preceding claims, the apparatus including a display device configured to present a user interface, wherein the microprocessor unit is further coupled to the display device.

8. The apparatus of any one of the preceding claims, wherein the data storage device is included in at least one of:
the sensory unit; and
an external computing device.

9. The apparatus of any one of the preceding claims, wherein the sensory unit is configured to generate a signal to the user, and wherein the microprocessor is configured to:
determine that the shock absorption performance data is outside one or more predetermined thresholds; and
in response to determining that the shock absorption performance data is outside the one or more predetermined thresholds, cause the sensory unit to generate the signal, wherein the signal indicates to the user that they are outside the predetermined thresholds.

10. The apparatus of claim 9, wherein the signal includes at least one of:
an audible signal; and
a tactile signal.

11. A method for monitoring shock absorption performance of a user using an apparatus for providing feedback to a user to optimize lower limb and core shock absorption performance, the apparatus including a sensory unit, a microprocessor unit, a data storage device and a communications unit, the method including:
collecting, by the sensory unit, shock absorption data representing shock absorption by hard tissues of the user while the user is wearing the apparatus;
generating, by the microprocessor unit, shock absorption performance data by processing the collected shock absorption data; and
transmitting, by the communication unit, the shock absorption performance data to a user device, wherein the user device is configured to execute a feedback module that receives and interprets the transmitted shock absorption performance data in order to generate feedback that can be provided to optimize the shock absorption performance of the user when the user is walking, running or rehabilitating.

12. The method of claim 11, wherein the method includes storing, by the data storage device, the shock absorption performance data.

13. The method of claim 11 or 12, the method including fixing or attaching the apparatus to the user's shinbone such that the sensory unit can collect the shock absorption data.

14. The method of any one of claims 11 to 13, the method including the microprocessor unit:
determining that the generated shock absorption performance data is outside one or more predetermined thresholds; and
in response to determining that the shock absorption performance data is outside one or more predetermined thresholds, causing the sensory unit to generate the signal, wherein the signal indicates to the user that they are outside predetermined thresholds.

15. The method of claim 14, wherein the signal includes at least one of:
an audible signal; and
a tactile signal.

16. A method for monitoring shock absorption performance of a user, the method including a user device executing a feedback module to:
receive shock absorption performance data transmitted to the user device;
generate feedback to be provided to the user to optimize the user's lower limb and core shock absorption, wherein generating the feedback is based at least in part on the received shock absorption data; and
providing the feedback to the user via a user interface of the user device.

17. The method of claim 16, the method including the user device executing the feedback module to:
receive, via the user interface, at least one of user details and user parameters,
wherein generating the feedback is based at least in part on the received user details and user parameters.

18. The method of claim 16 or 17, wherein the generated feedback includes at least one of:
feedback for the user to alter a current movement or exercise being performed by the user; and
feedback to modify an existing stored program or exercise protocol of the user.

19. The method of any one of claims 16 to 18, wherein the feedback includes at least one strengthening exercise, and providing the feedback to the user via the user interface includes displaying on the user interface one or more of:
a description of the exercise,
a picture or video of the exercise, and
an indication of the duration, repetitions, intensity and/or resistance of the exercise.

20. The method of any one of claims 16 to 19, the method including the user device executing the feedback module to store the generated feedback in a data store.

21. The method of any one of claims 16 to 20, the method including the user device executing the feedback module to:
identify, based at least in part on the received shock absorption performance data, a strike zone of the user;
determine that the identified strike zone is an inappropriate strike zone; and
in response to determining that the identified strike zone is the inappropriate strike zone, generate feedback prompting the user in relation to the user's foot strike.

22. A system including:
a wearable unit, for attaching to the medial ankle of a user, including a wireless transceiver, an accelerometer and an inertial measurement unit (IMU) to detect movement when walking and shock absorption by the ankle/lower limb of the user; and
a mobile device for communication with the wearable unit and including code of an application to execute at least some of the following:
a. access and display physio assessment questions for the user to answer;
b. process answers to the questions and display a series of objective movement tests for the user, such as squat and calf raise;
c. generate a user interface asking the user in response to the tests to mark on the user interface points of pain and score that pain;
d. generate and display instructions for the user to walk and obtain data from the wearable unit to generate user parameters relating to how the user moves when walking, such as shock absorption, knee data, step and speed;
e. analyse the user parameters obtained and generate recommendations for the user;
f. generate, based on analysis of the user parameters, coaching instructions to provide parameters for movement and a proper technique for walking, and generate and send commands to the wearable unit to guide the user, such as by haptics; and
g. generate weight loss instructions to guide the user to walk to produce optimal shock absorption to increase muscle activity (recruitment and contraction) and energy expended.
